(19) United States
(12) Patent Application Publication
(10) Pub. No.: US 2016/0222371 A1
(43) Pub. Date: Aug. 4, 2016

(54) CARBONIC ANHYDRASE WITH STABILITY AT HIGH TEMPERATURE AND CAPTURRING AGENT FOR CARBON DIOXIDE COMPRISING THE SAME

(71) Applicant: POSTECH ACADEMY-INDUSTRY FOUNDATION, Pohang (KR)

(72) Inventors: Hyung Joon CHA, Pohang (KR); Byung Hoon Jo, Seongnam (KR); Jeong Hyun Seo, Pohang (KR)

(21) Appl. No.: 15/131,146

(22) Filed: Apr. 18, 2016

Related U.S. Application Data
(63) Continuation of application No. PCT/KR2014/004328, filed on May 14, 2014.

(30) Foreign Application Priority Data
Oct. 17, 2013 (KR) 10-2013-0124221

(51) Int. Cl. C12N 9/88; B01D 53/84; B01D 53/62

(52) U.S. Cl. CPC C12N 9/88; B01D 53/62; B01D 53/84; C12Y 402/01001; B01D 2255/804

(57) ABSTRACT

The present invention relates to a carbonic anhydrase, a nucleic acid molecule encoding the carbonic anhydrase, a recombinant vector including the nucleic acid molecule, a host cell transformed with the recombinant vector, and a method of preparing the carbonic anhydrase using the host cell. The carbonic anhydrase of the present invention has an excellent stability at high temperature to exhibit a carbon dioxide capturing activity even at high temperature, thereby being applied to a carbon dioxide capturing process performed at high temperature with many advantages in view of economic aspect due to mass-production of expression system.

### CROSS-REFERENCES TO RELATED APPLICATION

This application is a Continuation Application of a National Stage application of PCT/KR2014/004328 filed on May 14, 2014, which claims priority to Korean Patent Application No. 10-2013-0124221 filed on Oct. 17, 2013, the contents of which are incorporated herein by reference.

### TECHNICAL FIELD

The present invention relates to a carbonic anhydrase having excellent stability at high temperature and a capturing agent for carbon dioxide including the same, wherein the carbonic anhydrase has an excellent stability at high temperature to maintain carbon dioxide capturing activity even at high temperature, thereby being applied to a carbon dioxide capturing process performed at high temperature.

### BACKGROUND ART

In accordance with steady increase in fossil energy use, concentration of carbon dioxide in atmosphere has increased, and with respect to global warming caused by the increased concentration of carbon dioxide, an effort to gradually decrease the concentration of carbon dioxide has increasingly accelerated. In addition to development of environment-friendly new renewable energy, technology of capturing and storing carbon dioxide in order to suppress rapid increase of carbon dioxide according to constantly increased fossil fuel use has received a lot of attention. To capture carbon dioxide, there are various methods such as a chemical absorption method and a physical absorption method, and the like. However, these methods have problems such as corrosion, high thermal energy, and the like. Living organisms have enzymes in which carbon dioxide is capable of being rapidly converted, and a biomimetic carbon dioxide capturing method using the enzymes has received attention as an environment-friendly technology of decreasing carbon dioxide. The enzyme is a carbonic anhydrase, and rapidly promotes a hydration reaction of carbon dioxide as a metalloenzyme containing zinc ions. The enzyme has intensively been researched in mammals since the mid-1900s, and it has been found that the enzyme is present in substantially almost all living organisms including bacteria and archaebacteria, and performs a variety of physiological roles such as photosynthesis, respiration, maintenance of homeostasis, bio-mineral formation, and the like.

Carbon dioxide in a gas form is dissolved in water, and this carbon dioxide forms carbonic acid by the hydration reaction. Under proper pH conditions, carbonate ions (CO₃²⁻) are finally formed. Then, the carbonate ions may react with metal cations to form a solid precipitate which is a carbonate mineral. The entire reaction is shown as the following Chemical Formulas 1 to 5.

CO₂(g)→CO₂(aq) [Chemical Formula 1]

CO₂(aq)+H₂O→H₂CO₃ [Chemical Formula 2]

H₂CO₃→H⁺+HCO₃⁻ [Chemical Formula 3]

HCO₃⁻→H⁺+CO₃²⁻ [Chemical Formula 4]

CO₃²⁻+Ca²⁺→CaCO₃ [Chemical Formula 5]

The hydration reaction of carbon dioxide dissolved in solution of Chemical Formula 2 is a rate-limiting step of Chemical Formulas 1 to 5, and the carbonic anhydrase catalyzes conversion of carbon dioxide and water into bicarbonate ions (HCO₃⁻) and hydrogen ions (H⁺), and therefore, a rate of the reaction is promoted up to ten million times faster than that of a natural reaction. Therefore, the carbon dioxide dissolved in a solution is capable being rapidly captured through the carbonic anhydrase, and this capturing g process includes the capturing into bicarbonate ions, and the conversion into carbonate minerals according to application.

This biomimetic technology using the carbonic anhydrase is environment-friendly and effective, however, there are still problems to be solved in actual processes, that is, reduction in production cost of the carbonic anhydrase and securement of enzyme stability. When the carbonic anhydrase is applied to the capturing process, a large amount of carbonic anhydrase should be obtained at a low price. The carbonic anhydrase extracted from bovine serum which has been mainly used in research has a price of about two million won per g, which is difficult to be applied in the actual process due to a high-priced problem. In addition, it is expected that the capturing process is applicable to power plants, steel mills, and the like, which discharge a large amount of carbon dioxide, wherein a heat cooling process of a flue gas containing carbon dioxide is required, and heat is released in an absorption column by carbon dioxide entering the absorption column while being melted. Further, in order to separate the melted and entering carbon dioxide as a gaseous state, a regeneration column needs to be maintained at high temperature. In order to apply the carbonic anhydrase to the absorption column process, fundamentally, the carbonic anhydrase needs to be stable at about 40 to 60° C., and in order to apply the carbonic anhydrase to the regeneration column process, activity needs to be maintained at higher temperature for a long time.

### SUMMARY OF INVENTION

### Technical Problem

The present invention has been made in an effort to provide a carbonic anhydrase, a nucleic acid molecule encoding the carbonic anhydrase, a recombinant vector including the nucleic acid molecule, a host cell transformed with the recombinant vector, and a method of preparing a carbonic anhydrase, using the host cell, in order to produce a carbonic anhydrase having high stability at high temperature and carbon dioxide capturing activity.

However, technical problems to be achieved in the present invention are not limited to the above-mentioned problems, and non-described other problems will be clearly understood to those skilled in the art from the following descriptions.

### Solution to Problem

The present invention provides a carbonic anhydrase, a nucleic acid molecule encoding the carbonic anhydrase, a recombinant vector including the nucleic acid molecule, a host cell transformed with the recombinant vector, and a method of preparing a carbonic anhydrase, using the host cell.

An exemplary embodiment of the present invention provides a carbonic anhydrase.

Another exemplary embodiment of the present invention provides a nucleic acid molecule encoding the carbonic anhydrase.

Still another exemplary embodiment of the present invention provides a recombinant vector including the nucleic acid molecule.

Still another exemplary embodiment of the present invention provides a host cell transformed with the recombinant vector.

Still another exemplary embodiment of the present invention provides a capturing agent for carbon dioxide including the carbonic anhydrase.

Still another exemplary embodiment of the present invention provides a method of preparing a carbonic anhydrase, using the host cell.

The present inventors searched carbonic anhydrase gene from genome information of thermophilic bacteria found in ocean floor fissure, constructed a recombinant expression vector based on the genome information, successfully mass-produced the carbonic anhydrase in Escherichia coli, and completed the present invention. It was confirmed that a lysate from a cell in which a recombinant carbonic anhydrase was expressed had high activity in c capturing carbon dioxide, and kinetic parameter of a purified carbonic anhydrase was more excellent than the known carbonic anhydrase derived from archaebacteria, and had an enzyme activity even at a temperature of 95° C., and even greater activity was shown under high temperature condition of the carbon dioxide capturing process as compared to at room temperature. In addition, the expressed carbonic anhydrase may maintain most of activities at high temperature and may have significantly high stability.

### Advantageous Effects of Invention

The carbonic anhydrase according to the present invention may have excellent stability at high temperature to exhibit a carbon dioxide capturing activity even at high temperature, thereby being applied to a carbon dioxide capturing process which is actually performed at high temperature. In addition, since mass-production is possible by using an expression system, it is expected that the carbonic anhydrase according to the present invention gives many advantages in view of economic aspect.

### BRIEF DESCRIPTION OF DRAWINGS

FIG. 1 illustrates results obtained by expressing carbonic anhydrases according to Example 2 in cytoplasm and analyzing the expressed carbonic anhydrases by SDS-PAGE.

FIG. 2 illustrates results obtained by measuring carbon dioxide capturing activities of the carbonic anhydrase, using lysates of host cells according to Example 3.

FIG. 3 illustrates results obtained by separating and purifying carbonic anhydrases according to Example 4, respectively, and analyzing the expressed carbonic anhydrases by SDS-PAGE.

FIG. 4 illustrates results obtained by measuring stabilities of purified carbonic anhydrases according to Example 5 at high temperature of 70° C.

FIGS. 5A and 5B are graphs illustrating stabilities of Persephonella marina-derived carbonic anhydrase and Thermovibrio ammonificans-derived carbonic anhydrase, measured at high temperature of 40° C. and 60° C. depending on time passage, respectively.

FIG. 6 is a graph illustrating activity changes of purified carbonic anhydrases according to Example 7 depending on temperature.

FIG. 7 is a graph showing the stability in aqueous amine solvent under high temperature (60° C.) condition.

### DETAIL DESCRIPTION

Hereinafter, the present invention will be described in detail.

An exemplary embodiment of the present invention provides a carbonic anhydrase.

The carbonic anhydrase may be derived from thermophilic bacteria, and has a carbon dioxide capturing activity maintained even at high temperature since it is derived from thermophilic bacteria. For example, the carbonic anhydrase may be derived from T. ammonificans, P. marina, or C. mediatlanticus, preferably, T. ammonificans.

The carbonic anhydrase is an enzyme having a molecular weight of about 27 kDa, and a reaction rate constant is KM=10 to 38 mM, and Kcat=2.8×10⁵ to 6.8×10⁵/s, respectively, using Lineweaver-Burk plot. In addition, the carbonic anhydrase has a specificity in which ester in addition to carbon dioxide is capable of being decomposed, and has an activity of 0.9 to 3.2 mol p-nitrophenyl acetate/mol enzyme·min.

The carbonic anhydrase is a thermostable enzyme, which maintains an activity even at 80° C. for at least 15 minutes, and maintains an enzyme activity even at 95° C. for a few minutes. Specifically, a carbon dioxide capturing activity may be 60% or more, preferably 70% or more, the most preferably 80% or more at 40 to 70° C. based on 100% of the carbon dioxide capturing activity at 4° C. Here, the carbon dioxide capturing activity means an activity in which the carbonic anhydrase converts carbon dioxide and water into bicarbonate ions (HCO₃⁻) and hydrogen ions (H⁺).

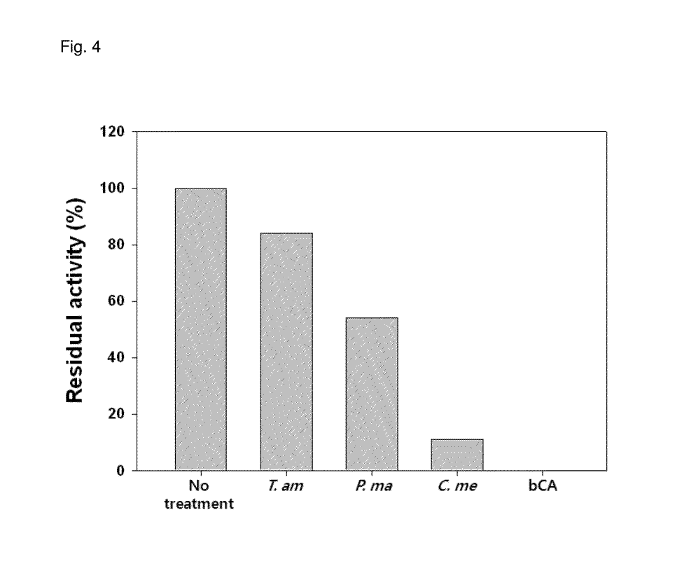

It was confirmed that T. ammonificans-derived carbonic anhydrase of the present invention had excellent stability at high temperature as compared to P. marina-derived carbonic anhydrase or C. mediatlanticus-derived carbonic anhydrase (FIGS. 4, 5, and 6).

The carbonic anhydrase may consist of an amino acid sequence of SEQ ID NO: 1, or the carbonic anhydrase may further include a restriction enzyme recognition site and/or an oligopeptide for purification at a terminal of the amino acid sequence of SEQ ID NO: 1. The restriction enzyme recognition site may include various sequences so as to match with a restriction enzyme site of a vector to be used when producing a recombinant enzyme, and as the oligopeptide for purification, a variety of Tags may be used, for example, 6×(His) tag. The carbonic anhydrase according to an exemplary embodiment of the present invention may be a peptide including an amino acid sequence of SEQ ID NO: 2 which is a peptide in which the restriction enzyme recognition site and/or the oligopeptide for purification are/is bound to a terminal of the amino acid sequence of SEQ ID NO: 1. In the present invention, the carbonic anhydrase of SEQ ID NO: 1 is derived from T. ammonificans, and the carbonic anhydrase of SEQ ID NO: 3 is derived from P. marina, which are compared with the carbonic anhydrase of SEQ ID NO: 4 derived from C. mediatlanticus.

Another exemplary embodiment of the present invention provides a nucleic acid molecule encoding the carbonic anhydrase.

The nucleic acid molecule may be obtained by removing signal base sequence of carbonic anhydrase from thermophilic bacteria. When including the signal base sequence, the carbonic anhydrase moves to a cell gap, such that expression is not sufficiently achieved in an expression system such as E. coli. The nucleic acid molecule may be appropriately controlled in consideration of codon bias in a host cell, preferably, E. coli.

The nucleic acid molecule may include a base sequence encoded by the amino acid sequence of SEQ ID NO: 1. Otherwise, the nucleic acid molecule may be a nucleic acid molecule encoded by the amino acid sequence further including the restriction enzyme recognition site and/or the oligopeptide for purification at a terminal of the amino acid sequence of SEQ ID NO: 1, and for example, may be a nucleic acid molecule encoded by the amino acid sequence of SEQ ID NO: 2.

Still another exemplary embodiment of the present invention provides a recombinant vector including the nucleic acid molecule. It means that the vector typically includes a transfer DNA into which foreign DNA is possible to be inserted, and as a kind of a nucleic acid molecule, the vector is bound to other different nucleic acid and transferred to a host cell, then expresses a target protein. For example, the vector includes all general vectors including a plasmid vector, a cosmid vector, a bacteriophage vector, a virus vector, and the like.

Still another exemplary embodiment of the present invention provides a host cell transformed with the recombinant vector. The recombinant vector may be introduced into the host cell, and may be introduced by performing known methods such as an electric shock gene transfer method (electroporation), calcium phosphate (CaPO₄) precipitation, or methods using calcium chloride (CaCl₂) precipitation, PEG, dextran sulfate, lipofectamine, and the like.

The host cell may be a prokaryotic cell. The prokaryotic cell is possible as long as it is a prokaryotic cell capable of being transformed with a foreign gene, and for example, the prokaryotic cell may include various microorganisms such as Escherichia coli, Rhodococcus, Pseudomonas, Streptomyces, Staphylococcus, Syfolobus, Thermoplasma, Thermoproteus, and the like, preferably, may be selected from the group consisting of Escherichia coli and Saccharomyces cerevisiae.

Preferably, the prokaryotic cell may be Escherichia coli, specifically, may include Escherichia coli XL1-blue, Escherichia coli BL21 (DE3), Escherichia coli JM109, Escherichia coli DH series, Escherichia coli TOP10, Escherichia coli HB101, and the like.

Still another exemplary embodiment of the present invention provides a method of preparing a carbonic anhydrase, using the host cell.

The transformed host cell may be incubated by appropriately controlling conditions such as medium ingredients, incubation temperature, incubation time, and the like. Specifically, a culture medium may contain all nutrients which are essential to growth and survival of microorganisms, such as carbon sources, nitrogen sources, trace element ingredients, and the like. PH of the medium may be appropriately controlled and may include ingredients such as antibiotics, and the like.

In addition, expression of the carbonic anhydrase may be induced by treating inducers such as isopropyl-β-D-thiogalactopyranoside (hereinafter, referred to as IPTG), and the like. Kinds of the inducer to be treated may be determined depending on the vector system, and conditions such as an administration time of the inducer, an administration amount of the inducer, and the like may be appropriately controlled. Conditions such as medium ingredients, incubation temperature, incubation time, and the like, may be appropriately determined depending on the kinds of the host cell to be used.

The expressed carbonic anhydrase may be recovered and purified by general methods. For example, cells recovered by centrifugation may be disrupted by French press, sonicator, and the like. When the carbonic anhydrase is secreted into an incubation liquid, an incubation supernatant may be gathered.

When aggregation occurs by overexpression, the carbonic anhydrase may be dissolved in a suitable solution to be denatured, followed by re-folding. Here, oxidation and reduction systems of glutathione, dithiothreitol, β-mercaptoethanol, β-mercaptomethanol, cystine, and cystamine may be used, and the re-folding agents may be urea, guanidine, arginine, and the like. Some of salts may be used together with the re-folding agents.

Here, a heat treatment process at 70 to 85° C. for 10 to 60 minutes may be added, and production scale of the carbonic anhydrase may be controlled so as to meet purposes.

Still another exemplary embodiment of the present invention provides a capturing agent for carbon dioxide including the carbonic anhydrase. The capturing agent may further include materials known as a capturing agent for carbon dioxide, for example, may include ammonia aqueous solution, alkanolamines or their aqueous solution including monoethanolamine (MEA), diethanolamine (DEA), methyldiethanolamine (MDEA), 2-amino-2-hydroxymethyl-1,3-propanediol (Tris), diglycolamine (DGA), 2-amino-2-methyl-1-propanol (AMP), 2-amino-2-hydroxymethyl-1,3-propanediol (AHPD), diisopropanol amine (DIPA), aqueous soluble salts (e.g. sodium or potassium salts) of N-methylaminopropionic acid or N,N-dimethylaminoacetic acid or N-methylalanine, N-methylglycine, beta-alanine (3-aminopropanoic acid) or other natural or modified amino acids (e.g. N-substituted amino acid derivatives), 2-(2-aminoethylamino)ethanol (AEE), triethanolamine (TEA) or other primary, secondary, tertiary or hindered amine-based solvents, potassium carbonate aqueous solution, and the like.

In conventional method, the concentration of capturing agent such as alkanolamine is typically 15 to 30% (v/v), or preferably at a lower concentration such as preferably below 15% (V/V). In generally, if the concentration is higher, the capturing efficiency becomes higher. However, the conventional enzyme cannot be stable in the concentration of capturing agent.

In an embodiment of the present invention, the carbonic anhydrase is preferably with the capturing agent at a concentration of capturing agent of higher than 30% (v/v), for example 35% (v/v) to 65% (v/v), 40% (v/v) to 65% (v/v), 35% (v/v) to 55% (v/v), or 40% (v/v) to 55% (v/v).

Still another exemplary embodiment of the present invention provides a method of capturing carbon dioxide, using the carbonic anhydrase. Specifically, the method may be a method of capturing carbon dioxide at 40 to 70° C., using the carbonic anhydrase.

Hereinafter, the present invention will be described in more detail by the following Examples. However, the following Examples are provided by way of example, and the scope of the present invention is not limited thereto.

### EXAMPLE

### Example 1

### Preparation of Transformed Host Cell

Nucleic acids encoding T. ammonificans-derived carbonic anhydrase, P. marina-derived carbonic anhydrase, and C. mediatlanticus-derived carbonic anhydrase based on NCBI database of genetic information were amplified by using PCR primers for genomic nucleic acid of each microorganism as a template, respectively.

Specifically, for expression of the carbonic anhydrase in cytoplasm, a base sequence of the carbonic anhydrase from which a signal sequence of T. ammonificans was removed, and a base sequence of the carbonic anhydrase from which a signal sequence of P. marina was removed were amplified, and for expression of C. mediatlanticus, a base sequence of the carbonic anhydrase from which a signal sequence of C. mediatlanticus was removed was amplified, and used primers were as follows. (Underlines in the following primer sequences mean restriction enzyme recognition sites)

Template for amplification of T. ammonificans carbonic anhydrase: Genomic DNA of Thermovibrio ammonificans (DSM 15698; gene accession number: WP_013538320)

A pair of primers for amplification of T. ammonificans carbonic anhydrase

Forward Primer
(SEQ ID NO: 5)
5′-ATACATATGGGTGGAGGAGCCCA-3′
Reverse Primer
(SEQ ID NO: 6)
5′-ATACTCGAGCTTCATAACCTTCCTTGCATT-3′

Template for amplification of P. marina carbonic anhydrase: Genomic DNA of Persephonella marina (DSM 14350; gene accession number: WP_015898908)

A pair of primers for amplification of P. marina carbonic anhydrase

Forward Primer
(SEQ ID NO: 7)
5′-ATACATATGGGTGGTGGCTGGAG-3′
Reverse Primer
(SEQ ID NO: 8)
5′-ATACTCGAGTTTTTCCATAATCATTCTTGCATTTAAAG-3′

Template for amplification of C. mediatlanticus carbonic anhydrase: Genomic DNA of Caminibacter mediatlanticus (DSM 16658; gene accession number: WP_007474387)

A pair of primers for amplification of C. mediatlanticus carbonic anhydrase

Forward Primer
(SEQ ID NO: 9)
5′-ATACATATGGGC TATAATTATCATGCAACTTGGAGTTATA-3′
Reverse Primer
(SEQ ID NO: 10)
5′-ATACTCGAGTTTTAAAATAACCCTTGCATTAATTGG-3′

Each amplification product was introduced into pET-22b (+) vector using NdeI, XhoI restriction enzymes, to finally construct three expression vectors, wherein each carbonic anhydrase gene in the pET-22b (+) vector has a histidine tag to be coupled with Ni ions at a C-terminal. Specifically, the T. ammonificans carbonic anhydrase has an amino acid sequence of SEQ ID NO: 2, the P. marina carbonic anhydrase has an amino acid sequence of SEQ ID NO: 3, and the C. mediatlanticus carbonic anhydrase has an amino acid sequence of SEQ ID NO: 4.

Each vector prepared by a heat shock method at 42° C. for 2 minutes was introduced into Escherichia coli BL21 (DE3) which is a host cell, and each host cell having the vector introduced thereinto was selected in an LB medium to which ampicillin was added, and three kinds of host cells expressing carbonic anhydrase derived from different microorganisms were finally prepared.

### Example 2

### Protein Expression Analysis

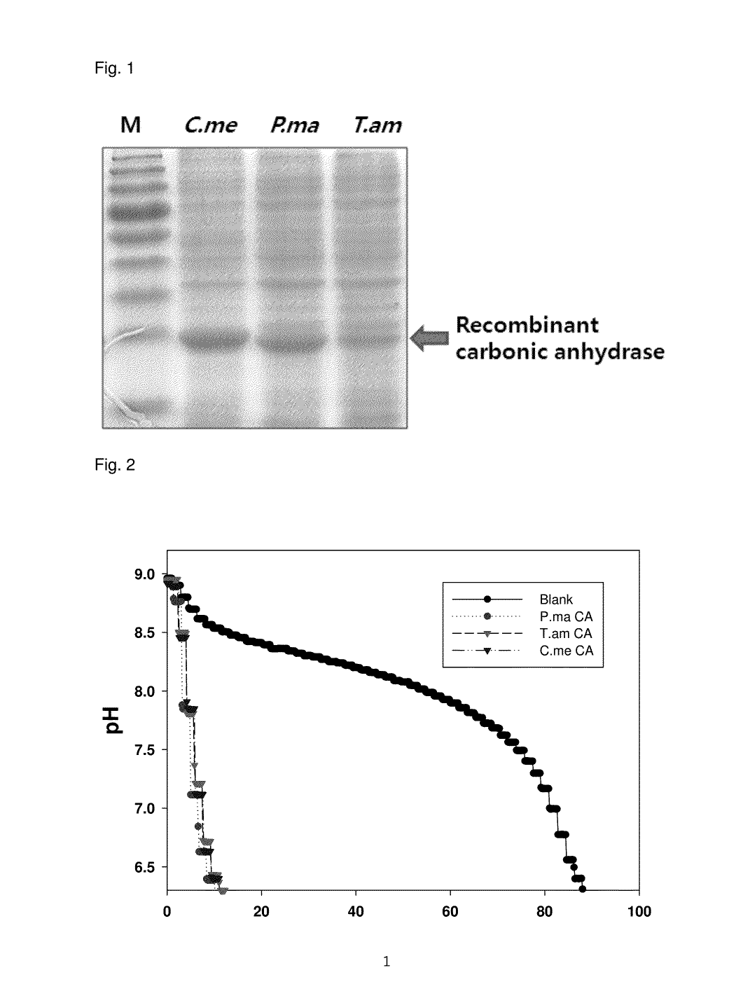

Each host cell prepared by Example 1 was incubated at 37° C. in an LB medium containing 50 μg/mL ampicillin added thereto, and when an absorbance (OD₆₀₀) of an incubation liquid was about 0.6 to 0.8, isopropyl-β-D-thiogalactopyranoside (IPTG, 1 mM) was added as an inducer to induce protein expression. After adding the IPTG, additionally, the host cells were incubated at 37° C. for 12 hours, then the incubated host cells were centrifuged at 4,000×g for 10 minutes, and a supernatant was removed and the host cells were recovered. The recovered host cells were suspended in a solution (50 mM sodium phosphate buffer, 300 mM NaCl, pH 8, 10 mM imidazole) for lysate, and were disrupted by sonicator. Then, total proteins of the disrupted host cells were analyzed by SDS-PAGE, and results thereof were shown in FIG. 1.

FIG. 1 illustrates results obtained by expressing three kinds of carbonic anhydrases in cytoplasm and analyzing the expressed carbonic anhydrases by SDS-PAGE, wherein M is a molecular weight standard marker, C.me is Caminibacter mediatlanticus-derived carbonic anhydrase, P.ma is Persephonella marina-derived carbonic anhydrase, and Tam is Thermovibrio ammonificans-derived carbonic anhydrase.

As shown in FIG. 1, it was confirmed that the carbonic anhydrase was successively expressed in each host cell.

### Example 3

### Confirmation of Carbon Dioxide Capturing Activity

Carbon dioxide capturing activities of each expressed carbonic anhydrase were measured by using the lysates of the host cells of Example 2. 100 μL of lysates of the host cells were added to 3 mL of 20 mM Tris sulfate buffer (pH 8.3), each CO₂ saturated H₂O solution was added thereto to thereby start a reaction, and it was observed that pH of each carbonic anhydrase was reduced, and results thereof were shown in FIG. 2.

FIG. 2 illustrates results obtained by measuring carbon dioxide capturing activities of three kinds of carbonic anhydrases, using lysates of the host cells, wherein Blank is a comparative group which does not include the carbonic anhydrase, P. ma CA is Persephonella marina-derived carbonic anhydrase, T. am CA is Thermovibrio ammonificans-derived carbonic anhydrase, and C. me CA is Caminibacter mediatlanticus-derived carbonic anhydrase.

As shown in FIG. 2, each pH of the Persephonella marina-derived carbonic anhydrase, the Thermovibrio ammonificans-derived carbonic anhydrase, and the Caminibacter mediatlanticus-derived carbonic anhydrase was rapidly decreased as compared to the comparative group, which means that the carbon dioxide capturing activity of the carbon anhydrase is high.

### Example 4

### Purification of Carbonic Anhydrase

A lysate of the host cell of Example 2 was centrifuged at 10,000×g for 20 minutes, and the carbonic anhydrase of supernatant was separated and purified. Specifically, a supernatant thereof was applied to a column filled with nickel resin so that the carbonic anhydrase was bound to the column, and carbonic anhydrase which was not bound to the column was washed with a wash buffer (50 mM sodium phosphate buffer, 300 mM NaCl, 30 mM imidazole, pH 8.0). Elution of the carbonic anhydrase from the column was performed using 50 mM sodium phosphate buffer, 300 mM NaCl, 250 mM imidazole (pH 8.0), then the purified solution was changed to 20 mM Tris-sulfate (300 mM NaCl, pH 8.3) using dialysis, and imidazole was removed. Three kinds of carbonic anhydrases were finally purified.

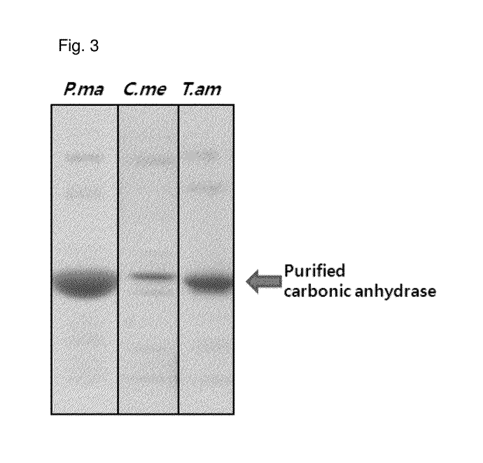

The purified three kinds of carbonic anhydrases were analyzed by SDS-PAGE as shown in Example 2, and results thereof were shown in FIG. 3.

FIG. 3 illustrates results obtained by separating and purifying three kinds of carbonic anhydrases, and analyzing the carbonic anhydrases by SDS-PAGE, wherein P. ma is Persephonella marina-derived carbonic anhydrase, C. me is Caminibacter mediatlanticus-derived carbonic anhydrase, and T. am is Thermovibrio ammonificans-derived carbonic anhydrase.

As shown in FIG. 3, it was confirmed that each carbonic anhydrase was completely purified by SDS-PAGE analysis.

### Example 5

### Comparison in View of Stability at High Temperature

In order to confirm stability of the carbonic anhydrase at high temperature, three kinds of the purified carbonic anhydrases that were separated and purified in Example 4 were allowed to stand at 70° C. for 16 hours, and reduction degree of activity was measured while comparing with an experimental group (shown by no treatment in FIG. 4) that was stored at 4° C., wherein a commercially available carbonic anhydrase derived from bovine serum was used as a control group. Activities were measured according to measurement of carbon dioxide capturing activity used in Example 3, and results thereof were shown in FIG. 4.

FIG. 4 illustrates results obtained by measuring stabilities of three kinds of purified carbonic anhydrases at high temperature of 70° C., wherein no treatment is a non-heated control group, T. am is Thermovibrio ammonificans-derived carbonic anhydrase, P. ma is Persephonella marina-derived carbonic anhydrase, C. me is Caminibacter mediatlanticus-derived carbonic anhydrase, bCA is bovine serum-derived carbonic anhydrase, a residual activity (%) is an activity which is maintained at the time of performing heat treatment as compared to a case in which an initial heat treatment was not performed.

As shown in FIG. 4, it was confirmed that Thermovibrio ammonificans-derived carbonic anhydrase showed 80% or more of residual activity, such that stability was maintained even at high temperature.

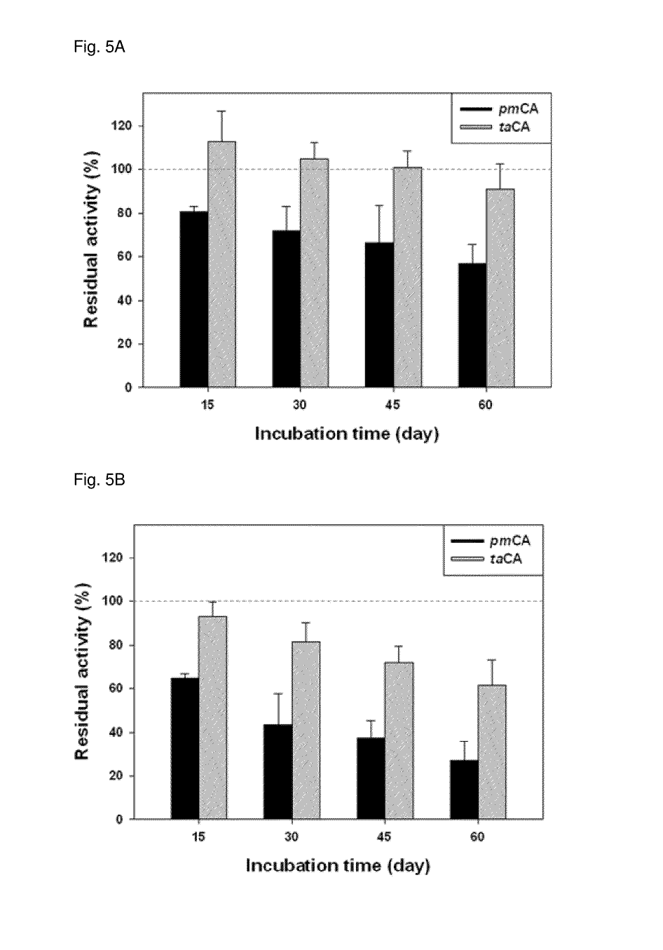

In addition, in order to confirm thermal stability for a long time under actual capturing temperature condition, thermal stability was tested at 40° C. and 60° C. for 60 days. Similarly, a reduction degree of activity was measured by allowing the enzymes to stand at each temperature for a predetermined time and comparing the enzymes with the experimental group stored at 4° C., and results thereof were shown in FIG. 5.

FIGS. 5A and 5B are graphs illustrating stabilities of Persephonella marina-derived carbonic anhydrase and Thermovibrio ammonificans-derived carbonic anhydrase, measured at high temperature of 40° C. and 60° C. depending on time passage, respectively.

As shown in FIGS. 5A and 5B, high thermal stability was shown in the Thermovibrio ammonificans-derived carbonic anhydrase and the Persephonella marina-derived carbonic anhydrase. In particular, the Thermovibrio ammonificans-derived carbonic anhydrase had 91% of an initial activity after 60 days at 40° C. (FIG. 5A), and even at 60° C., 62% of an initial activity after 60 days was maintained (FIG. 5B).

### Example 6

### Measurement of Kinetic Parameter

In order to conduct accurate kinetic measurement with respect to CO₂, stopped-flow spectroscopy was used. 100 mM N-Tris(hydroxymethyl)methyl-3-aminopropanesulfonic acid (TAPS)/NaOH buffer (which includes 57.2 mM Na₂SO₄ and 97.2 μM m-cresol purple, pH 8.5) including 10 nM to 100 nM of carbonic anhydrase was mixed with various concentrations of CO₂ solutions, and initial pH change was observed by absorbance change at 578 nm at 25° C. K_{M} and K_{cat} values were obtained from the obtained data by Michaelis-menten Equation.

The carbonic anhydrase had a capability as an esterase which is capable of decomposing ester in addition to a substrate specificity with respect to CO₂. 100 μl of 30 mM p-nitrophenyl acetate was added to a solution including 800 μl of buffer (50 mM potassium phosphate; pH 7.0) and 100 μl of the carbonic anhydrase, and mixed well, and absorbance change at 348 nm at 25° C. for 3 minutes was measured to observe an esterase activity. Table 1 shows kinetic parameters of bCA, T. am CA, and P. ma CA. Table 1 shows reaction rate values of the purified carbonic anhydrases according to Example 6.

TABLE 1

| | Esterase | | | |
|---|---|---|---|---|
| | activity | CO₂ hydration activity | | |
| Classification | (min⁻¹) | k_{cat} (s⁻¹) | K_{M} (mM) | k_{cat}/K_{M} (M⁻¹ × s⁻¹) |
| bCA | 46.9 | 5.8 × 10⁵ | 14.3 | 4.1 × 10⁷ |
| pmCA | 3.2 | 2.8 × 10⁵ | 10.3 | 2.7 × 10⁷ |
| taCA | 0.9 | 6.8 × 10⁵ | 37.9 | 1.8 × 10⁷ |

### Example 7

### Activity Change Depending on Temperature

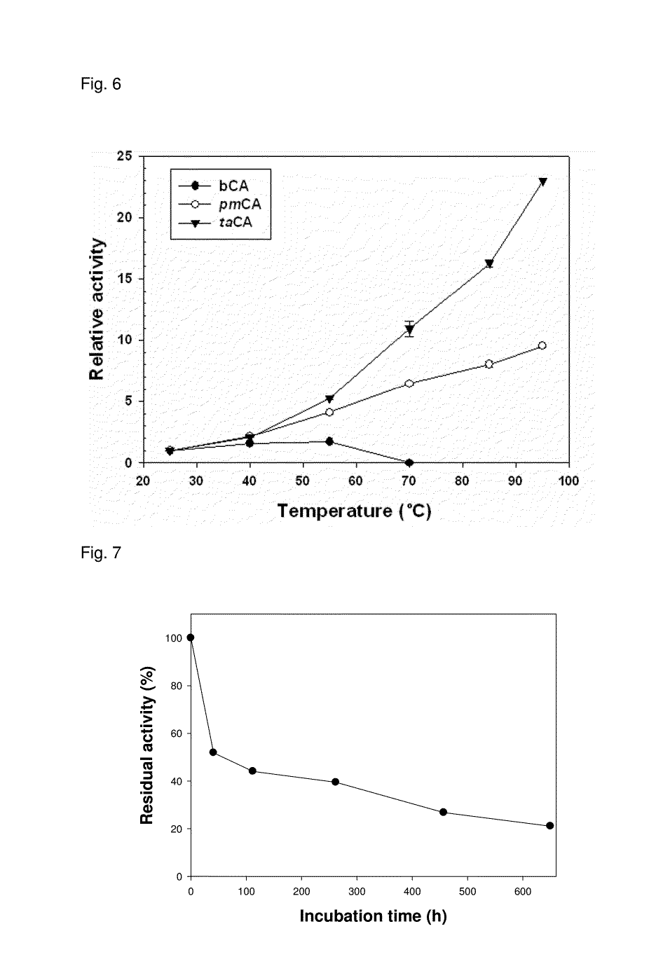

Activity change of the carbonic anhydrase depending on temperature was measured by applying the measurement of esterase activity used in Example 6 at higher temperatures rather than 25° C. As shown in FIG. 6, both of P. marina-derived and T. ammonificans-derived carbonic anhydrases had much higher activity at high temperature. The activities were the highest at 95° C., and at a temperature higher than 95° C., the activities could not be measured due to experiment characteristic. In consideration of optimal growth temperature (around 70° C.) of the two microorganisms, it was assumed that optimum reaction temperature of the two carbonic anhydrases were not significantly higher than 95° C.

### Example 8

### Stability in Aqueous Amine Solvent Under High Temperature Condition

Stability of the carbonic anhydrase in aqueous N-methyl diethanolamine (MDEA) solvent was estimated by incubating the T. ammonificans-driven purified carbonic anhydrases obtained in Example 4 under 4.2M MDEA (50% v/v) at 60° C. followed by measurement of residual activity of the incubated enzyme using initial CO₂ hydration activities obtained by stopped-flow spectroscopic technique used in Example 6.

As shown in FIG. 7, T. ammonificans-derived carbonic anhydrase showed high stability under the specified condition. Even though the enzyme showed the initial drastic drop of the residual activity after about incubation for 40 hours, further decrease was slow showing the overall half-life of ˜15 days for the enzyme inactivation.

The above description of the present invention is provided for illustrative purposes, and it will be understood to those skilled in the art that the exemplary embodiments can be easily modified into various forms without changing the technical spirit or essential features of the present invention. Accordingly, the exemplary embodiments described herein are provided by way of example only in all aspects and should not be construed as being limited thereto.

1. A carbonic anhydrase derived from Thermovibrio ammonificans, having a carbon dioxide capturing activity of 60% or higher at 40 to 70° C. based on 100% of the carbon dioxide capturing activity at 4° C.

2. The carbonic anhydrase of claim 1, wherein the carbon dioxide capturing activity of the carbonic anhydrase is 70% or higher.

3. The carbonic anhydrase of claim 1, wherein the carbonic anhydrase is a peptide including an amino acid sequence of SEQ ID NO: 1.

4. The carbonic anhydrase of claim 3, wherein the carbonic anhydrase further comprises at least one selected from the group consisting of a restriction enzyme recognition site and an oligopeptide for purification, connected to a C-terminus of the peptide comprising an amino acid sequence of SEQ ID NO: 1.

5. The carbonic anhydrase of claim 4, wherein the carbonic anhydrase is a peptide comprising an amino acid sequence of SEQ ID NO: 2.

6. A nucleic acid molecule encoding the carbonic anhydrase of claim 1.

7. The nucleic acid molecule of claim 6, wherein the nucleic acid molecule consists of nucleic acid sequences encoding an amino acid sequence of SEQ ID NO: 1 or an amino acid sequence of SEQ ID NO: 2.

8. A recombinant vector comprising an nucleic acid molecule of claim 6 or 7.

9. The recombinant vector of claim 8, wherein the recombinant vector is selected from the group consisting of a plasmid vector, a cosmid vector, a bacteriophage vector, and a virus vector.

10. A host cell transformed with the recombinant vector of claim 8.

11. The host cell of claim 10, wherein the host cell is a prokaryotic cell.

12. A capturing agent for carbon dioxide comprising:
the carbonic anhydrase of claim 1, a microorganism including the carbonic anhydrase, a lysate of the microorganism, or an extract of the lysate of the microorganism.

13. A method of capturing carbon dioxide at 40 to 60° C., using the carbonic anhydrase of claim 1.

14. The method of claim 13, wherein the carbon dioxide capturing activity of the carbonic anhydrase is 70% or higher.

15. The method of claim 13, wherein the carbonic anhydrase is a peptide including an amino acid sequence of SEQ ID NO: 1.

16. The method of claim 15, wherein the carbonic anhydrase further comprises at least one selected from the group consisting of a restriction enzyme recognition site and an oligopeptide for purification, connected to a C-terminus of the peptide comprising an amino acid sequence of SEQ ID NO: 1.

17. The method of claim 16, wherein the carbonic anhydrase is a peptide comprising an amino acid sequence of SEQ ID NO: 2.

18. The method of claim 1, wherein method is performed with a second capturing agent at a concentration of second capturing agent of higher than 30% (v/v).

19. The method of claim 18, wherein the second capturing agent is selected from the group consisting of ammonia aqueous solution, alkanolamine aqueous solution and potassium carbonate aqueous solution.

20. The method of claim 19, wherein the alkanolamine is monoethanolamine (MEA), diethanolamine (DEA), methyldiethanolamine (MDEA), 2-amino-2-hydroxymethyl-1,3-propanediol (Tris), diglycolamine (DGA), 2-amino-2-methyl-1-propanol (AMP), 2-amino-2-hydroxymethyl-1,3-propanediol (AHPD), diisopropanol amine (DIPA), aqueous soluble salts (e.g. sodium or potassium salts) of N-methylaminopropionic acid or N,N-dimethylaminoacetic acid or N-methylalanine, N-methylglycine, beta-alanine (3-aminopropanoic acid) 2-(2-aminoethylamino)ethanol (AEE), or triethanolamine (TEA).

21. The method of claim 18, wherein the concentration of second capturing agent ranges from 35% (v/v) to 65% (v/v).


SEQUENCE LISTING (10 sequences)

SEQ ID NO: 1 - protein, 228 aa, Thermovibrio ammonificans
MGGGAHWGYSGSIGPEHWGDLSPEYLMCKIGKNQSPIDINSADAVKACLAPVSVYYVSDAKYVVNNGHTIKVVMGGRGYVVVDGKRFYLKQFHFHAPSEHTVNGKHYPFEAHFVHLDKNGNITVLGVFFKVGKENPELEKVWRVMPEEPGQKRHLTARIDPEKLLPENRDYYRYSGSLTTPPCSEGVRWIVFKEPVEMSREQLEKFRKVMGFDNNRPVQPLNARKVMK

SEQ ID NO: 2 - protein, 236 aa, Thermovibrio ammonificans
MGGGAHWGYSGSIGPEHWGDLSPEYLMCKIGKNQSPIDINSADAVKACLAPVSVYYVSDAKYVVNNGHTIKVVMGGRGYVVVDGKRFYLKQFHFHAPSEHTVNGKHYPFEAHFVHLDKNGNITVLGVFFKVGKENPELEKVWRVMPEEPGQKRHLTARIDPEKLLPENRDYYRYSGSLTTPPCSEGVRWIVFKEPVEMSREQLEKFRKVMGFDNNRPVQPLNARKVMKLEHHHHHH

SEQ ID NO: 3 - protein, 233 aa, Persephonella marina
MGGGWSYHGEHGPEHWGDLKDEYIMCKIGKNQSPVDINRIVDAKLKPIKIEYRAGATKVLNNGHTIKVSYEPGSYIVVDGIKFELKQFHFHAPSEHKLKGQHYPFEAHFVHADKHGNLAVIGVFFKEGRENPILEKIWKVMPENAGEEVKLAHKINAEDLLPKDRDYYRYSGSLTTPPCSEGVRWIVMEEEMEMSKEQIEKFRKIMGGDTNRPVQPLNARMIMEKLEHHHHHH

SEQ ID NO: 4 - protein, 239 aa, Caminibacter mediatlanticus
MGYNYHATWSYSGKTGPEYWGDLKKEYQMCKIGKNQSPIDIKTNSTQTFNTNLKPFKIKYLGKGYEVINNGHTIKVKTEGKNCVRIDGIKFKLAQLHFHTPSENTINGKHFPMEAHYVHLDKNGNITVLAVMYKIGKENKSLNKMLAVLPTKVGEENKVMGNLNPMELLPKNKAYYRFNGSLTTPPCSEGVRWIVFKTPVEISQAQYEKMHAVMGTNNRPVQPINARVILKLEHHHHHH

SEQ ID NO: 5 - DNA, 23 bp, Artificial Sequence
  Forward primer for amplifying the carbonic anhydrase of T. ammonificans
atacatatgggtggaggagccca

SEQ ID NO: 6 - DNA, 30 bp, Artificial Sequence
  Reverse primer amplifying carbonic anhydrase of T. ammonificans
atactcgagcttcataaccttccttgcatt

SEQ ID NO: 7 - DNA, 23 bp, Artificial Sequence
  Forward primer amplifying carbonic anhydrase of P. marina
atacatatgggtggtggctggag

SEQ ID NO: 8 - DNA, 38 bp, Artificial Sequence
  Reverse primer amplifying carbonic anhydrase of P. marina
atactcgagtttttccataatcattcttgcatttaaag

SEQ ID NO: 9 - DNA, 40 bp, Artificial Sequence
  Forward primer amplifying carbonic anhydrase of C. mediatlanticus
atacatatgggctataattatcatgcaacttggagttata

SEQ ID NO: 10 - DNA, 36 bp, Artificial Sequence
  Reverse primer amplifying carbonic anhydrase of C. mediatlanticus
atactcgagttttaaaataacccttgcattaattgg